(19) United States
(12) Patent Application Publication
(10) Pub. No.: US 2007/0055195 A1
(43) Pub. Date: Mar. 8, 2007

(54) Hand held ultraviolet blood purifier

(76) Inventor: Warren G. Browne, Philadelphia, PA (US)

Correspondence Address: ZACHARY T. WOBENSMITH, III, 7746 101st Court, Vero Beach, FL, 32967-2871, US

(21) Appl. No.: 11/218,215

(22) Filed: Sep. 2, 2005

(51) Int. Cl. A61M 37/00

(52) U.S. Cl. 604/6.08

(57) ABSTRACT

A hand held ultraviolet blood purifier system is disclosed, which has an outer housing having two battery operated ultraviolet light sources mounted thereto, with a removable syringe therebetween, the syringe having a blood repository and treatment chamber, a plunger and a hollow needle for withdrawing and returning blood.

### BACKGROUND OF THE INVENTION

1. Field of the Invention

This invention relates to a hand held ultraviolet blood purifier system of the portable self contained type.

2. Description of the Prior Art

In the prior art, it has been known to use ultraviolet light to purify water and other fluids. Most of the systems using ultraviolet light are stationary flow-through systems which include a chamber having an ultraviolet light source with a fluid inlet and outlet. The fluid to be treated flows through the chamber past the ultraviolet light source, which destroys the various viruses, bacteria and some other contaminants. It is desirable to be able to treat the blood of a person, whose blood is contaminated with viruses, and bacteria, some of which may be resistant to antibiotics. It is also desirable to be able to treat blood specimens in a laboratory, to determine if various viruses and bacteria respond to exposure to ultraviolet light and are destroyed.

A system or device to be useful must be hand held, portable, self contained, and easily used by a person.

In treating the blood of a person it is desirable to be able to withdraw a portion of a person's blood supply, treat it and then return it to the person.

No suitable system or device is currently available that fulfills the requirements.

Likewise, no suitable system or device is currently available for use in a laboratory to treat blood with ultraviolet light to destroy viruses and bacteria.

The hand held system of the invention is suitable to treat a person's blood, and also treat blood specimens in a laboratory.

### SUMMARY OF THE INVENTION

A hand held ultraviolet blood purifier system is provided which is self contained, has two ultraviolet light sources with a replaceable syringe therebetween, is battery operated, is useful to treat the blood of a person and blood specimens in a laboratory.

The principal object of the invention is to provide a hand held self-contained ultraviolet blood purifier system.

A further object of the invention is to provide a blood purifier system that is battery operated.

A further object of the invention is to provide a blood purifier system that is easily carried on and by an individual.

A further object of the invention is to provide a blood purifier system that may be used to treat a person's blood or to treat blood specimens in a laboratory.

A further object of the invention is to provide a blood purifier system that is simple and inexpensive to construct,. and lends itself to mass production.

A further object of the invention is to provide a blood purifier system that has a disposable syringe.

A further object of the invention is to provide a blood purifier system that is durable and long lasting in use.

Other objects and advantageous features of the invention will be apparent from the description and claims.

### DESCRIPTION OF THE DRAWINGS

The nature and characteristic features of the invention will be more readily understood from the following description taken in connection with the accompanying drawings forming part thereof in which:

FIG. 1 is a perspective view of a blood purifier system in accordance with the invention;

FIG. 2 is a top plan view of the system of FIG. 1;

FIG. 3 is a vertical sectional view taken approximately on the line 3-3 of FIG. 2;

FIG. 4 is a horizontal sectional view taken approximately on the line 4-4 of FIG. 3, and

FIG. 5 is a schematic of the electrical circuitry of the system of the invention.

It should, of course, be understood that the description and drawing herein are merely illustrative, and that various modifications and changes can be made in the structures, and embodiments disclosed without departing from the spirit of the invention.

### DESCRIPTION OF THE PREFERRED EMBODIMENT

When referring to the preferred embodiment, certain terminology will be utilized for the sake of clarity. Use of such terminology is intended to encompass not only the described embodiment, but also technical equivalents, which operate and function in substantially the same way to bring about the same result.

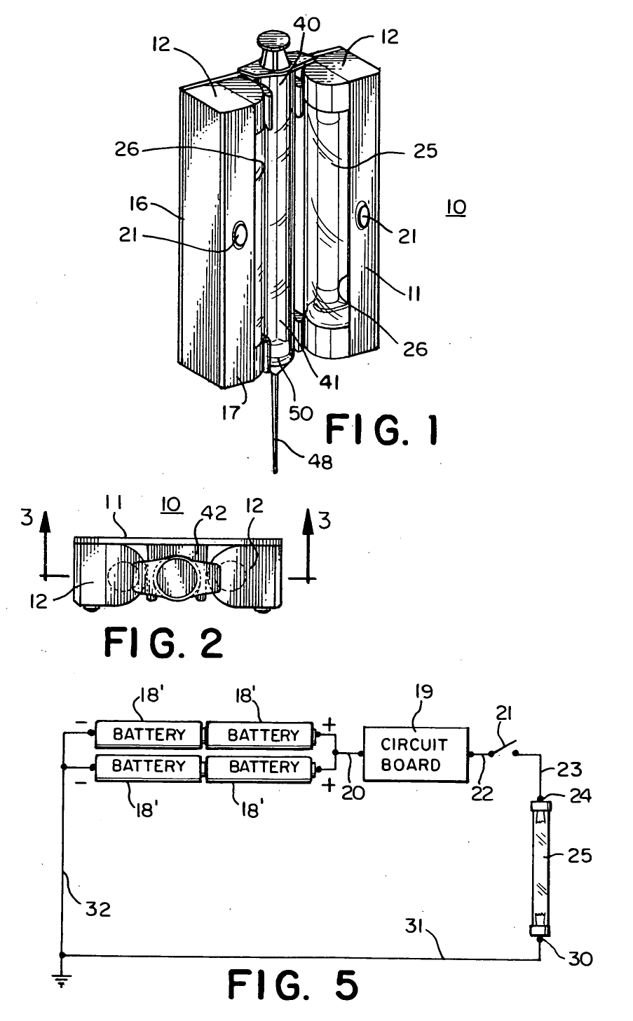

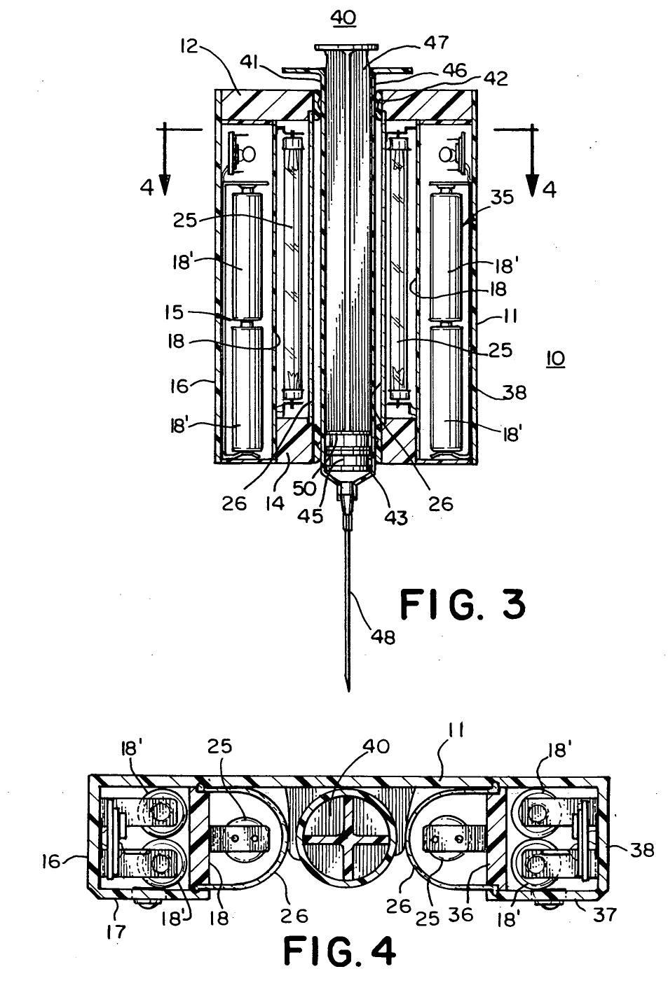

Referring now to the drawings and FIG. 1-4, a hand held ultraviolet blood purifier system 10 is therein illustrated. The purifier system 10 has an outer housing 11, which can be of synthetic plastic, polyethylene plastic being particularly suitable. The outer housing 11 is of rectangular configuration with a top plate 12 and bottom plate 14. The housing 11 as shown at the left in FIGS. 1 and 3 has a compartment 15, with a side wall 16, front wall 17 and right side wall 18. The compartment 15 has a plurality of batteries 18′ therein, four being illustrated of well known type. The batteries 18′ as shown in FIG. 5 are series/parallel connected on the positive side to a circuit board 19, of well known type, by wire 20. The circuit board 19 is connected to a switch 21 by wire 22, with a wire 23 connecting the switch 21 to one side 24 of an ultraviolet bulb 25, which is mounted to side wall 18 and has a removable transparent semi-circular cover 26 therearound.

The ultraviolet bulb 25 is of well known type, and available from Top Bulb, 5204 Indianapolis Boulevard, East Chicago, Ind., 46312-3838. The other side 30 of bulb 25 is connected by wire 31 to ground G, with a wire 32 connected to the ground side of the batteries 18′.

Referring to the right side of system 11, another compartment 35 is provided, having a left side wall 36, a front wall 37 and a right side wall 38. A plurality of batteries 18′ are mounted in compartment 35, with the identical circuitry as described for compartment 15. An ultraviolet light bulb 25 is mounted to the left side wall 36, with a removable transparent cover 26 around light bulb 25.

Between the transparent covers 26 a syringe 40 is provided, which is of cylindrical shape, with a transparent outer housing 41, carried in a syringe harness 42 in the top plate 12, and a syringe harness 43 in bottom plate 14. The syringe harnesses 42 and 43 are preferably of O-ring configuration, and grip the outer housing 41, but permit the syringe 40 to be removed as required. The syringe 40 includes a plunger 45, finger grips 46 at the top 47, and has a hollow syringe needle 48.

The plunger 45 is in housing 41, which forms a blood repository and disinfection chamber 50 in housing. 41.

In use, the ultraviolet bulbs 25 are activated, the syringe needle 48 is inserted into a person's vein, and the plunger 45 withdrawn, causing blood (not shown) to be drawn up into the chamber 50, where it is subject to exposure to ultraviolet light.

When treatment is completed, depending on the blood contaminants, the plunger 45 is pushed back in the housing 41, forcing the treated blood back into the person's vein. The operation can continue as desired. After use, the syringe 40 can be removed from the outer housing 11, and discarded in a chemotherapeutic waste container, a new syringe 40 can be inserted and a new patient treated.

It should be noted that the needle 48 can also be inserted into a blood sample (not shown) to be treated, and the treated blood then returned as desired.

It will thus be seen that the objects of the invention have been achieved.

I claim:

1. A hand held ultraviolet blood purifier system which comprises,
an outer housing,
said outer housing having compartments at each side,
a plurality of batteries in said compartments,
electrical means in said compartment connected to said batteries,
ultraviolet light sources connected to said electrical means, external to and mounted to said compartments,
said ultraviolet light sources having transparent covers therearound and,
syringe means mounted in said housing between said ultraviolet light sources.

2. A hand held ultraviolet blood purifier system as defined in claim 1 in which,
said electrical means includes a circuit board and an on-off switch.

3. A hand held ultraviolet blood purifier system as defined in claim 1, in which;
said syringe means includes,
an outer housing having a blood repository and disinfection chamber,
a plunger in said chamber for drawing blood thereinto, and
a hollow needle in communication with said chamber permitting blood flow into and out of said chamber.

4. A hand held ultraviolet blood purifier system as defined in claim 1 in which;
said syringe means is removable.
